(19) United States
(12) Patent Application Publication
(10) Pub. No.: US 2016/0000661 A1
(43) Pub. Date: Jan. 7, 2016

(54) NO-RINSE HAIR CLEANSING FORMULATION AND WIPES

(71) Applicant: Essential Effects LLC, Beverly Hills, CA (US)

(72) Inventors: Ron P. Van, Los Angeles, CA (US); Joshua A. Pineiro, Los Angeles, CA (US); Kim Wu, Los Angeles, CA (US); Harry Wu, Los Angeles, CA (US)

(73) Assignee: Essential Effects LLC, Beverly Hills, CA (US)

(21) Appl. No.: 14/789,568

(22) Filed: Jul. 1, 2015

Related U.S. Application Data
(60) Provisional application No. 61/998,589, filed on Jul. 2, 2014.

(51) Int. Cl. A61K 8/02; A61K 8/55; A61K 8/86; A61K 8/40; A61K 8/97; A61K 8/46; A61K 8/895; A61Q 5/02; A61K 8/67; A61K 8/49; A61K 8/34

(52) U.S. Cl. CPC A61K 8/0208; A61K 8/4946; A61K 8/55; A61K 8/86; A61K 8/40; A61K 8/34; A61K 8/466; A61K 8/895; A61Q 5/02; A61K 8/67; A61K 8/97; A61K 2800/594; A61K 2800/87

(57) ABSTRACT

A no-rinse cleansing composition comprising a formulation comprising one or more ingredients of Table 1 or one or more ingredients of Table 2 or one or more ingredients of Table 3 and a substrate to remove soiled substance.

### CROSS-REFERENCES TO RELATED APPLICATIONS

This application claims the benefit of U.S. Provisional Application No. 61/998,589 filed Jul. 2, 2014, which is incorporated herein by reference.

### BACKGROUND OF THE INVENTION

This invention relates to the field of personal hygiene products, and more specifically, to hair cleansing disposable wipes for both humans and pets. The invention comprises of a no-rinse hair cleansing solution integrated with non-woven disposable wipes to create hair cleansing wipes that effectively cleans and refreshes human hair and pet hair without the use of water for rinsing.

Accordingly, the present invention provides an easy and convenient way to clean and refresh hair. For example, it is the ideal solution for seniors at all stages, those who are entrusted to caregivers taking care of our seniors and those with limited mobility, people who are unable to wash their hair due to surgery, frequent travelers and backpackers, military personnel, and those who simply are short on time or find themselves in a situation where access to water is limited. Shampoo wipes are also a perfect way to keep hair feeling clean and fresh in between washes.

There are vast arrays of personal hygiene products currently being marketed and sold as hair cleansers and shampoos. These cleansers and shampoos, which are typically in liquid form, instruct the consumer to apply the product directly to damp hair, then lather and rinse. More recently, there is recognition that not all consumers, for one reason or another, are able to clean their hair in this traditional manner. In addition, there are differing opinions as to whether daily washing of hair is actually healthy for the hair and scalp. As a result, “dry” shampoos have been developed. These dry shampoo products, which are applied as an aerosol spray, while more convenient, do not leave hair feeling clean and fresh due to the powdery residue that is left in the hair. In fact, this residue must be combed out of the hair.

Historically, talcum powders and starches have been used to absorb, remove and clean oily substances secreted from the sebaceous gland in the hair follicles, combination of cell debris, sweat, and dust from the air that make the hair look and feel greasy. It was used on patients in hospitals, to clean and refresh hair pieces and wigs in Europe as early as the late 18^{th} and early 19^{th} centuries.

One personal hygene product currently on the market is dry shampoo. The first commercially available aerosol dry shampoo was available in 1940 and became popular again in 2012-2014. The main ingredients of dry shampoo are starches from corn, rice and the like, in an aerosol and powder form. However, there are distinct disadvantages associated with the use of dry shampoo, including an unpleasant chalky feel of the hair after use and the residual starches leave hair white, and aerosol particles created impact respiratory issue.

Another existing product is a shampoo cap is in the form of a pre-formed shower cap, containing a diluted form of shampoo solution and materials that hold the solution. To use the shampoo cap, a user puts the cap over the head and rubs the hair to clean. Again, certain disadvantages are apparent with the shower cap. For example, it is expensive for a single use product and it leaves hair feeling greasy.

Shampoo gloves are similar to the shampoo cap, in that the glove works similar to the shampoo cap, but is in the form of a glove.

Additionally, there are also many different types of cleansing wipes being marketed and sold at the present time, ranging from hand sanitizing wipes to face and body cleansing wipes. However, none of those wipes are able to cleanse and freshen hair.

The foregoing examples of the related art and limitations are intended to be illustrative and not exclusive. Other limitations of the related art will become apparent to those of skill in the art upon a reading of the specification and a study of the drawings or figures as provided herein.

### SUMMARY OF THE INVENTION

Accordingly, the above issues and disadvantage are obviated by the present invention which provides a novel, more convenient way to clean hair by using a disposable pre-moistened wipe with extremely mild cleansing solution which not only cleans the hair but at the same time, conditions and refreshes the hair, leaving the hair silky smooth, soft and manageable.

In one aspect of the present invention, there is provided a no-rinse cleansing composition comprising a formulation, wherein the formulation comprises the ingredients of Table 1, the ingredients of Table 2, or the ingredients of Table 3, and a substrate to remove soiled substance.

In another aspect of the present application, the formulation as disclosed in the present application may comprise water as a solvent and ethyl alcohol as a co-solvent, lipophilic agent.

In another aspect of the present application, the cleansing formulation may be pre-absorbed in a nonwoven towellette substrate and packaged in a closed package.

In one embodiment, the cleansing formulation comprises water as a solvent, a mild non-sulfate surfactant, a conditioning agent, an anti-static agent, an alcohol, a fragrance and optionally a preservative to prevent microbial growth.

In one embodiment, the substrate may be a nonwoven substrate.

In one aspect of the present application, the cleansing composition may be enclosed in a package which may be a sealed laminated pouch.

In one embodiment, the substrate may be a nonwoven material which is fabricated by spun lace 50% PET and 50% Rayon materials. In another embodiment, the substrate may be a material which has high absorbency to retain the solution without dripping during use.

In another aspect, the shampoo wipe can be conveniently stored, carried and transported anywhere until it is opened and used.

In yet another aspect, the disposable towelette may be made out of non-woven material or out of any suitable substrates. In one embodiment, the towelette may be pre-moistened with a cleansing solution in a sealed package, for the purpose of cleaning soiled greasy hair resulting from natural oil from the sebaceous glands in the hair follicles, also cleans facial and body hair without further rinsing out with water.

In one aspect, the present invention combines the use of a no-rinse cleanser made specifically for hair, with non-woven disposable wipes, enabling easy and convenient application, while leaving hair feeling clean and naturally fresh. In one embodiment, the no-rinse cleanser may be specifically made for human hair. The present invention will benefit a wide segment of consumers, from those who are constantly on the go, to consumers who are confined to assisted living arrangements due to age or physical condition, as well as the healthcare professionals who are responsible for their daily care and well-being.

In addition to human consumer, another aspect of the present invention may also be made for animals or pets. In one embodiment, the “pet” version of this invention will help pet owners keep their furry loved-ones clean and fresh in between baths. In various embodiments, animals and pets comprise dogs, cats, or other domesticated pets. The domesticated animals and pets include animals and pets with fur.

The present invention will also further the goal of helping to preserve the environment by reducing the amount of water use.

In one aspect of the present application, there is provided a no-rinse hair cleansing formulation which may be combined with non-woven disposable wipes, enabling easy and convenient application, while leaving hair feeling clean and naturally fresh. In one embodiment, the no-rinse hair cleansing formulation may be specially formulated with gentle cleansing ingredients. In another embodiment, the no-rinse hair cleansing solution may be formulated with gentle cleansing ingredients, including among other things, plants and herbal extracts, vitamin complex blend, moisturizer and antistatic agents.

In one embodiment, the detailed listing of the chemical composition of the cleanser are disclosed in the Tables of Chemical Compounds, Tables 1 to 12. In another embodiment, the specially formulated cleanser does not contain any harsh chemicals. In one embodiment, the harsh chemicals may include chemicals such as sulfate surfactants, salt, parabens, gluten, or preservatives, which may damage or dry out the hair.

In another aspect of the invention, different cleansers may be formulated to account for different human hair types. In one embodiment, the human hair types comprise soft, coarse, fine, or thick hair types.

In one aspect of the present invention, the no-rinse cleanser will be infused or loaded into the non-woven disposable wipes during the manufacturing process. Therefore, the texture of the cleansing wipes will be soft and moist to the touch. In one embodiment, the wipes will be white in color.

The hair cleansing wipes are convenient and easy to use. The consumer will simply remove a cleansing wipe from the packaging. To use the wipe, the consumer will gently apply the wipe to the consumer's entire head of hair. For a thorough cleanse, the consumer should apply the wipe while running the fingers through the hair. In one embodiment, the consumer will not need to add water to activate the cleanser, nor will water be needed to rinse out the cleanser. Once the consumer has applied the wipe to his or her hair, the hair will be left to air dry, but may also be blow dried.

In one aspect of the present invention, the hair cleansing wipes will be developed in varying sizes and different weight configuration depending upon the targeted application. In one embodiment, generally, the human hair cleansing wipes may be rectangular or square in shape. In another embodiment, the cleansing wipes may be approximately the size of a paper towel and may be customized at the request of the consumer.

In another aspect of the present invention, the cleansing wipes may also be available in different light scents. In one embodiment, the scents comprise fragrances that are widely and commonly used in the manufacturing of shampoo, no-rinse shampoo, or other hair care product.

In another aspect of the present invention, the hair cleansing wipes for pets is expected to be larger in size due to greater variation in pet sizes. In one embodiment, the cleanser infused within the pet wipes will be specially formulated to be safe and suitable for pets.

The hair cleansing wipes will also come in different packaging depending upon the targeted application. Each packaging type will contain a different number of wipes. Hair cleansing wipes used at the commercial level will come in larger quantities while wipes packaged for end consumer use is expected to contain fewer wipes due to the smaller size of the packaging. The wipes will also be available in travel size.

In addition to the exemplary embodiments, aspects and variations described above, further embodiments, aspects and variations will become apparent by reference to the data and tables and by examination of the following descriptions.

### DETAILED DESCRIPTION OF THE INVENTION

Unless specifically noted otherwise herein, the definitions of the terms used are standard definitions used in the art of manufacturing shampoo such as no-rinse shampoo. Exemplary embodiments, aspects and variations are illustratived in the figures and drawings, and it is intended that the embodiments, aspects and variations, and the figures and drawings disclosed herein are to be considered illustrative and not limiting.

In one aspect of the present application, there is provided a no-rinse cleansing substrate comprising a composition, wherein the composition comprises a formulation comprising one or more ingredients of Table 1 or one or more ingredients of Table 2 or one or more ingredients of Table 3 or one or more ingredients of Table 12, wherein the substrate is used to clean hair.

In one embodiment, the formulation comprises water as a solvent and an alcohol such as a low molecular weight alcohol such as ethyl alcohol as a co-solvent, lipophilic agent.

In another aspect of the present application, there is provided a no-rinse cleansing composition comprising a formulation comprising one or more ingredients of Table 4, one or more ingredients of Table 5, one or more ingredients of Table 6, one or more ingredients of Table 7, one or more ingredients of Table 8, one or more ingredients of Table 9, one or more ingredients of Table 10, one or more ingredients of Table 11, or one or more ingredients of Table 12.

In yet another embodiment, the formulation comprises low molecular weight alcohol such as methyl (methanol), isopropyl and butyl alcohol. In various embodiments, the low molecular weight methyl, isopropyl and butyl alcohol may act as the co-solvent. Other similar low molecular weight alcohol may also function as a co-solvent. In another embodiment, the alcohol may aid to dissolve and remove any oily substance from the hair.

In another embodiment, the formulation may comprise a surface active agent. In one embodiment, the surface active agent is a sulfate free surfactant. In yet another embodiment, the surface active agent is Cocamindopropyl Hydrosultaine.

In another aspect of the present application, the formulation may comprise surface active agent which may be a sulfate based surfactant. Sulfate based surfactants are well known to one skilled in the art of formulating cleansing composition such as haircare product.

In one aspect, the formulation further comprises an anti-static agent. In one embodiment, the anti-static agent may comprise quaternium materials that exhibit cationic anti-static properties. In one embodiment, the anti-static agent is Quaternium 52.

In yet another aspect of the present application, the formulation comprises a conditioning agent. Various conditioning agents are well known to one in the art of manufacturing or formulating shampoo, including no-rinse shampoo. In one embodiment, the conditioning agent may be Divinyldimethicane/Dimethicone Copolymer. In yet another embodiment, the formulation comprises other silicone and long chain quaternium ammonium halogen compound.

In one aspect of the present invention, the formulation may be loaded onto a substrate. In one embodiment, the substrate is a wipe. In another embodiment, the substrate is a nonwoven wipe. In yet another embodiment, the nonwoven wipe may be a disposable wipe.

In one embodiment, the nonwoven wipe comprises a spunlace of 50% PET and 50% Rayon. In another embodiment, the substrate comprises materials such as PET, Rayon, woodpulp, bamboo rayon, and cotton. In another embodiment, the material for the substrate comprises paper or disposable nonwoven material of other polymeric components.

In order to maintain healthy hair, the composition may comprise a vitamin complex blend and natural extracts from plants. In another aspect, fragrances may be used to impart a pleasant, refreshing scent to the hair, or to mask undesirable odor.

In one embodiment, the vitamin complex blend comprises Biotin, other B-complex Vitamins, Vitamin A, Vitamin C, Vitamin H, Keratin, and Vitamin D3.

In one embodiment, the natural plant extract comprises Camellia Sinensis Extract, Citrus Aurantium Amara Peel Extract (Bitter Orange), Astragalus Root (Membranaceus) Extract (Milk Vetch), Schizandra Chinensis Fruit Extract, Pinus Tabulaeformis Bark Extract (Pine), Vitis Vinifera Seed Extract (Grape), White Willow extract, Sedum Rosea Root Extract, Rehmannia Chinensis Root Extract, Coconut oil, Aloe Vera or Aloe Vera Extract, Organic Shea butter, Beta Glucan, Organic tea tree oil, Pomegranate oil, Moringa seed extract, Soy amino acids, Rosemary oil, Neem oil, Green tea extract, Couch grass, Pineapple extract, Ginseng, Nettle and Coltsfoot extracts, Acacia extract, lemongrass extract, Noni Berry extract, Jasmine oil, Sesame extract, and Jojoba Oil.

In another embodiment, the natural plant extract comprise traditional Chinese herbal extracts. In yet another embodiment, the cleansing formulation comprises honey extract.

In yet various embodiments, the cleansing formulation disclosed herein comprises a biosurfactant. In one embodiment, the biosurfactant comprises from about 1 to 20% by weight of the cleansing formulation. In another embodiment, the biosurfactant comprises from about 2 to 18%, from about 3 to 16%, from about 5 to 15%, or from about 7 to 10% by weight of the cleansing formulation. The biosurfactants are well known to one in the art of manufacturing shampoo and may comprise glycolipids, phospholipids and fatty acids, lipopeptides and lipoproteins.

In another aspect of the present application, there is provided a cleansing kit, comprising the composition of the formulation enclosed in a sealed container. In one embodiment the seal container may be a sealed pouch. In yet another embodiment, the sealed pouch may comprise laminated flexible foil, wherein the foil is a 3-layer foil film comprising PET/AL6/LL60. The laminated flexible foil may be heat-sealed to contain the composition and to retain moisture.

In one aspect, the composition comprising the substrate (also called the shampoo wipe) is designed to clean hair, facial hair and body hair without further use of water to rinse out. In various embodiments of the composition, the ratio of the formulation and the substrate of nonwoven material is carefully measured to ensure there is no dripping during the entire application of the wipe.

In one aspect of the present application, the cleansing formulation displays surprising and unexpected efficacy when the formulation is applied by users with itchy, flaky scalp problems. The user's hair is cleansed effectively without the itchy and flaky scalp symptoms that accompany the use of regular shampoo.

In various aspect of the present application, the cleansing formulation exhibits advantageous property such as antiseptic properties which help to maintain the health of the scalp by preventing skin irritations, stimulating the growth of hair by keeping the scalp moisturized, or enhancing the natural shine and color of hair.

In one aspect of the present application, the size of the nonwoven wipe may be 12×10 inches and the thickness may be 70 grams per square meter. A 12×10 inches nonwoven wipe may be suitable for use on short to shoulder length hair; or for longer and fuller hair, two packages may be required. Other suitable wipe or substrate may have different sizes. In one embodiment, the size of the substrate may be 10×8, 9×7, 8×6, 7×7, 6×4, or 5×5 inches. In another embodiment, the thickness of the nonwoven substrate may be from about 30 to about 70, from about 40 to about 80, from about 50 to about 90, or from about 60 to about 100 grams per square meter.

In another embodiment, the no-rinse cleansing composition may comprise at least one of C12-C13 Pareth, C12-C13 Pareth 3, Diazolidinyl Urea, Iodopropynyl Butylcarbamate.

In one aspect of the present application, the water may act as the main solvent and carrier for other ingredients. In one embodiment, the special denatured ethanol may act as a co-solvent and solublizer for any oily substances on the hair.

In one embodiment, the non-sulfate surfactant may act to lower the surface tension of the oily substance on the hair, enabling the oily substance to be properly wiped off the hair and to be absorbed on the nonwoven wipe. In yet another embodiment, the formulation may comprise cationic quaternium conditioning agent.

In another embodiment, the no-rinse composition may also include silicone conditioner that improves wet and dry combing and leave the hair silky soft feel. Optionally, the no-rinse formulation may comprise a preservative to prevent microbial growth in case of any mishandling during manufacturing process.

In one aspect of the present application, there is provided a no-rinse shampoo wipe which is designed to clean hair without additional use of water besides the solution within the package.

In another aspect of the application, the ratio by weight of the formulation to the nonwoven material enable the use of the no-rinse wipe without drip during the cleaning process. In one embodiment, to prevent the formulation from dripping from the nonwoven substrate (a no drip application), the formulation may be loaded at a ratio of five (5) parts of solution by weight to one (1) part by weight of the nonwoven material. In another embodiment, the ratio by weight of formulation to substrate may be 1:1, 1.5:1, 2:1, 2.5:1, 3:1, 3.5:1, 4:1, 4.5:1, 5:1, 5.5:1, 6:1, 6.5:1, 7:1, 7.5:1, 8:1, 8.5:1, 9:1, 9.5:1, or 10:1.

In one aspect of the present application, a user can tear and open the package from the cut-out at the package, remove the pre-moistened wipe, apply directly to dry hair, gently massage hair with the wipe, starting at the root and working towards the ends of the hair. In one embodiment, the user should wipe the entire length of the hair thoroughly. The user may apply a new wipe to remove excess dirt and oil as needed. Additionally, the hair may be air dried, towel dried or blow dried on gentle heat setting after application of the no-rinse wipe.

In another aspect of the application, three basic components of the no-rinse composition comprise a solvent, a surface active agent, and a substrate to soak up the substance. In one aspect, the three basic components may comprise water as the solvent, a mild sulfate free surfactant as the surface active agent, and the nowoven wipe as the substrate to soak up and remove the soiled substance form the hair.

In yet another embodiment, the no-rinse composition may be enclosed in a package. The package may be a laminated pliable poly-plastic/aluminum foil which exhibits non-perforate properties. In one embodiment, the package is heat sealed air tight after the insertion of the nonwoven wipe and the solution to prevent evaporation during prolonged storage.

In one embodiment, the composition comprises water, alcohol, C12-C13 Pareth-23, C12-C13 Pareth-3, Cocamidopryl PC-Dimonium Chloride Phosphate, Cocoamindopropyl Hydroxysultaine, Diazolidinyl Urea, Iodopropynyl Butylcarbamate, Quaternium-52, Polyquaternium-4, Divinyldimethicone/Dimethicone Copolymer, fragrance, vitamin complex blend, and natural plant extract.

In another embodiment, the composition comprises water, alcohol, C12-C13 Pareth-23, C12-C13 Pareth-3, Cocamidopryl PC-Dimonium Chloride Phosphate, Cocoamindopropyl Hydroxysultaine, Diazolidinyl Urea, Iodopropynyl Butylcarbamate, Quaternium-52, Polyquaternium-4, Divinyldimethicone/Dimethicone Copolymer, vitamin complex blend, and natural plant extract.

In another embodiment, the composition comprises water, alcohol, C12-C13 Pareth-3, Cocamidopryl PC-Dimonium Chloride Phosphate, Cocoamindopropyl Hydroxysultaine, Diazolidinyl Urea, Iodopropynyl Butylcarbamate, Quaternium-52, Polyquaternium-4, Divinyldimethicone/Dimethicone Copolymer, vitamin complex blend, and natural plant extract.

In another embodiment, the composition comprises water, alcohol, C12-C13 Pareth-23, Cocamidopryl PC-Dimonium Chloride Phosphate, Cocoamindopropyl Hydroxysultaine, Diazolidinyl Urea, Iodopropynyl Butylcarbamate, Quaternium-52, Polyquaternium-4, Divinyldimethicone/Dimethicone Copolymer, vitamin complex blend, and natural plant extract.

In another embodiment, the composition comprises water, alcohol, C12-C13 Pareth-23, C12-C13 Pareth-3, Cocamidopryl PC-Dimonium Chloride Phosphate, Cocoamindopropyl Hydroxysultaine, Iodopropynyl Butylcarbamate, Quaternium-52, Polyquaternium-4, Divinyldimethicone/Dimethicone Copolymer, vitamin complex blend, and natural plant extract.

In another embodiment, the composition comprises water, alcohol, C12-C13 Pareth-23, C12-C13 Pareth-3, Cocamidopryl PC-Dimonium Chloride Phosphate, Cocoamindopropyl Hydroxysultaine, Diazolidinyl Urea, Quaternium-52, Polyquaternium-4, Divinyldimethicone/Dimethicone Copolymer, vitamin complex blend, and natural plant extract.

In another embodiment, the composition comprises water, alcohol, C12-C13 Pareth-23, C12-C13 Pareth-3, Cocamidopryl PC-Dimonium Chloride Phosphate, Cocoamindopropyl Hydroxysultaine, Quaternium-52, Polyquaternium-4, Divinyldimethicone/Dimethicone Copolymer, vitamin complex blend, and natural plant extract.

In another embodiment, the composition comprises water, alcohol, C12-C13 Pareth-23, C12-C13 Pareth-3, Cocamidopryl PC-Dimonium Chloride Phosphate, Cocoamindopropyl Hydroxysultaine, Diazolidinyl Urea, Iodopropynyl Butylcarbamate, Quaternium-52, Polyquaternium-4, Divinyldimethicone/Dimethicone Copolymer, and fragrances.

In another embodiment, the composition comprises water, alcohol, Cocamidopryl PC-Dimonium Chloride Phosphate, Cocoamidopropyl Hydroxysultaine, Diazolidinyl Urea, Iodopropynyl Butylcarbamate, Quaternium-52, Polyquaternium-4, Divinyldimethicone/Dimethicone Copolymer, and natural plant extract.

In another embodiment, the composition comprises water, alcohol, C12-C13 Pareth-23, C12-C13 Pareth-3, Cocoamindopropyl Hydroxysultaine, Diazolidinyl Urea, Iodopropynyl Butylcarbamate, Quaternium-52, Polyquaternium-4, Divinyldimethicone/Dimethicone Copolymer, vitamin complex blend, and natural plant extract.

In another embodiment, the composition comprises water, alcohol, Cocoamidopropyl Hydroxysultaine, Iodopropynyl Butylcarbamate, Quaternium-52, Polyquaternium-4, Divinyldimethicone/Dimethicone Copolymer, vitamin complex blend, and natural plant extract.

In another embodiment, the composition comprises water, alcohol, Cocoamindopropyl Hydroxysultaine, Polyquaternium-4, and natural plant extract.

As disclosed in all of the embodiments in the present application, the terms “formulation” and “cleansing formulation” may be used interchangeably.

As disclosed in all of the embodiments in the present application, the terms “composition” and “cleansing composition” may be used interchangeably.

TABLE 1
Compounds/Ingredients of no-rinse shampoo formulation

| | |
|---|---|
| Water | C12-C13 Pareth-23, C12-C13 Pareth-3 |
| Alcohol | Cocamidopryl PC-Dimonium Chloride |
| | Phosphate |
| Cocoamindopropyl | Diazolidinyl Urea, Iodopropynyl |
| Hydroxysultaine | Butylcarbamate |
| Quaternium-52 | Divinyldimethicone/Dimethicone Copolymer |
| Polyquaternium-4 | Vitamin complex blend |
| Fragrance | Natural plant extract |

TABLE 2
Compounds/Ingredients of no-rinse shampoo formulation

| | |
|---|---|
| Water | C12-C13 Pareth-23, C12-C13 Pareth-3 |
| Alcohol | Cocamidopryl PC-Dimonium Chloride |
| | Phosphate |
| Cocoamindopropyl | Diazolidinyl Urea, Iodopropynyl |
| Hydroxysultaine | Butylcarbamate |
| Quaternium-52 | Fragrance |
| Polyquaternium-4 | Natural plant extract |
| Divinyldimethicone/ | |
| Dimethicone Copolymer | |

TABLE 3
Compounds/Ingredients of no-rinse shampoo formulation

| | |
|---|---|
| Water | Divinyldimethicone/Dimethicone Copolymer |
| Alcohol | C12-C13 Pareth-23, C12-C13 Pareth-3 |
| Cocoamindopropyl | Cocamidopryl PC-Dimonium Chloride |
| Hydroxysultaine | Phosphate |
| Quaternium-52 | Diazolidinyl Urea, Iodopropynyl |
| | Butylcarbamate |
| Polyquaternium-4 | Fragrance |

TABLE 4
Compounds/Ingredients of no-rinse shampoo formulation

| | |
|---|---|
| Water | Divinyldimethicone/Dimethicone Copolymer |
| Alcohol | C12-C13 Pareth-23, C12-C13 Pareth-3 |
| Cocoamindopropyl | Cocamidopryl PC-Dimonium Chloride |
| Hydroxysultaine | Phosphate |
| Quaternium-52 | Diazolidinyl Urea |
| Polyquaternium-4 | Fragrance |

TABLE 5
Compounds/Ingredients of no-rinse shampoo formulation

| | |
|---|---|
| Water | Diazolidinyl Urea, Iodopropynyl |
| | Butylcarbamate |
| Alcohol | C12-C13 Pareth-23, C12-C13 Pareth-3 |
| Cocoamindopropyl | Divinyldimethicone/Dimethicone Copolymer |
| Hydroxysultaine | |
| Quaternium-52 | Cocamidopryl PC-Dimonium Chloride |
| | Phosphate |
| Polyquaternium-4 | |

TABLE 6
Compounds/Ingredients of no-rinse shampoo formulation

| | |
|---|---|
| Water | Polyquaternium-4 |
| Alcohol | Divinyldimethicone/Dimethicone Copolymer |
| Cocoamindopropyl | C12-C13 Pareth-23, C12-C13 Pareth-3 |
| Hydroxysultaine | |
| Quaternium-52 | Cocamidopryl PC-Dimonium Chloride |
| | Phosphate |

TABLE 7
Compounds/Ingredients of no-rinse shampoo formulation

| | |
|---|---|
| Water | Cocamidopryl PC-Dimonium Chloride |
| | Phosphate |
| Alcohol | Divinyldimethicone/Dimethicone Copolymer |
| Cocoamindopropyl | Polyquaternium-4 |
| Hydroxysultaine | |
| Quaternium-52 | |

TABLE 8
Compounds/Ingredients of no-rinse shampoo formulation

| | |
|---|---|
| Water | C12-C13 Pareth-23, C12-C13 Pareth-3 |
| Alcohol | Divinyldimethicone/Dimethicone Copolymer |
| Cocoamindopropyl | Polyquaternium-4 |
| Hydroxysultaine | |
| Quaternium-52 | |

TABLE 9
Compounds/Ingredients of no-rinse shampoo formulation

| | |
|---|---|
| Water | Divinyldimethicone/Dimethicone Copolymer |
| Alcohol | Cocoamindopropyl Hydroxysultaine |
| Quaternium-52 | Polyquaternium-4 |

TABLE 10
Compounds/Ingredients of no-rinse shampoo formulation

| | |
|---|---|
| Water | Quaternium-52 |
| Alcohol | Polyquaternium-4 |
| Cocoamindopropyl | |
| Hydroxysultaine | |

TABLE 11
Compounds/Ingredients of no-rinse shampoo formulation

| | |
|---|---|
| Water | Quaternium-52 |
| Cocoamindopropyl | Alcohol |
| Hydroxysultaine | |

TABLE 12
Compounds/Ingredients of no-rinse shampoo formulation

| | |
|---|---|
| Water | Cocoamindopropyl Hydroxysultaine |
| Alcohol | |

### Preparation of No-Rinse Hair Cleansing Substrate Integrated Various Composition

### Example 1

### Formula No. EE 14161-1, Compounds of the No-Rinse Cleansing Formulation

| Percent by weight | Mininum | Normal | Maximun |
|---|---|---|---|
| Water | 10.000 | 90.550 | 99.900 |
| Alcohol | 0.010 | 8.000 | 30.000 |
| Cocoamindopropyl Hydroxysultaine | 0.010 | 0.500 | 4.000 |
| Quaternium-52 | 0.010 | 0.250 | 5.000 |
| Polyquaternium-4 | 0.010 | 0.050 | 3.000 |
| Divinyldimethicone/Dimethicone | 0.010 | 0.500 | 5.000 |
| Copolymer | | | |
| C12-C13 Pareth-23, C12-C13 Pareth-3 | 0.010 | 0.380 | 4.000 |
| Cocamidopryl PC-Dimonium Chloride | 0.010 | 0.150 | 5.000 |
| Phosphate | | | |
| Diazolidinyl Urea, Iodopropynyl | 0.010 | 0.070 | 2.000 |
| Butylcarbamate | | | |
| Fragrance | 0.010 | 0.050 | 4.000 |
| Total | | 100.000 | |

### Example 2

### Formula No. EE 14161-2, Compounds of the No-Rinse Cleansing Formulation

| INGREDIENTS | WEIGHT (%) |
|---|---|
| Water | q.s. |
| Denatured Ethyl Alcohol | 6.0-15.0 |
| Quaternium-52 | 1.0-3.0 |
| Polyquaternium-4 | 0.5-2.0 |
| Cocamidoproply PG-Dimonium | 0.5-2.0 |
| Chloride Phosphate | |
| Coamidopropyl Hydroxysultaine | 0.5-3.0 |
| Fragrance | 0.1-2.0 |
| Plant and Herbal Extract | 0.1-2.0 |
| Panthenol | 0.1-2.0 |
| Vitamin complex blend | 0.1-2.0 |

### Example 3

### Formula No. EE 14161-3, Compounds of the No-Rinse Cleansing Formulation

| INGREDIENTS | WEIGHT (%) |
|---|---|
| Water | q.s. |
| Denatured Ethyl Alcohol | 10-20 |
| Quaternium-52 | 0.5-5 |
| Polyquaternium-4 | 2.5-4.5 |
| Cocamidoproply PG-Dimonium | 2.5-4.5 |
| Chloride Phosphate | |
| Coamidopropyl Hydroxysultaine | 4.5-7.5 |
| Fragrance | 0.5-3.5 |
| Plant and Herbal Extract | 0.7-3.5 |
| Panthenol | 0.5-3.5 |
| Vitamin complex blend | 2.5-4.5 |

### Example 4

### Formula No. EE 14161-4, Compounds of the No-Rinse Cleansing Formulation

| INGREDIENTS | WEIGHT (%) |
|---|---|
| Water | q.s. |
| Denatured Ethyl Alcohol | 15-35 |
| Quaternium-52 | 3.5-8.5 |
| Polyquaternium-4 | 4.5-6.5 |
| Cocamidoproply PG-Dimonium | 4.5-7.5 |
| Chloride Phosphate | |
| Coamidopropyl Hydroxysultaine | 5.5-9.5 |
| Fragrance | 2.5-5.5 |
| Plant and Herbal Extract | 5.5-7.5 |
| Panthenol | 4.5-6.5 |
| Vitamin complex blend | 4.5-6.5 |

### Example 5

### Formula No. EE 14161-5, Compounds of the No-Rinse Cleansing Formulation

| | Percentage ratio of each ingredient | | | | |
|---|---|---|---|---|---|
| INGREDIENTS | to the remaining ingredients | | | | |
| Water | 97 | 95 | 94 | 93 | 92 |
| Alcohol | 0.5 | 1 | 2 | 3 | 4 |
| Cocoamindopropyl | 0.1 | 0.2 | 0.3 | 0.4 | 0.5 |
| Hydroxysultaine | | | | | |
| Quaternium-52 | 0.01 | 0.03 | 0.06 | 1 | 1.5 |
| Polyquaternium-4 | 0.01 | 0.03 | 0.07 | 1 | 1.3 |
| Divinyldimethicone/Dimethicone | 0.01 | 0.05 | 0.2 | 0.3 | 0.7 |
| Copolymer, | | | | | |
| C12-C13 Pareth-23, C12-C13 | 0.01 | 0.05 | 0.1 | 0.5 | 1 |
| Pareth-3 | | | | | |
| Cocamidopryl PC-Dimonium | 0.01 | 0.05 | 0.2 | 0.3 | 0.7 |
| Chloride Phosphate | | | | | |
| Diazolidinyl Urea, Iodopropynyl | 0.01 | 0.05 | 0.2 | 0.3 | 0.5 |
| Butylcarbamate | | | | | |
| Fragrance | 0.01 | 0.05 | 0.1 | 0.3 | 0.5 |

### Example 6

### Formula No. EE 14161-6, Compounds of the No-Rinse Cleansing Formulation

| | Percentage ratio of each ingredient | | | | | |
|---|---|---|---|---|---|---|
| INGREDIENTS | to the remaining ingredients | | | | | |
| Water | 91 | 90 | 89 | 88 | 87 | 86 |
| Alcohol | 5 | 6 | 7 | 8 | 9 | 10 |
| Cocoamindopropyl | 1 | 1.5 | 2 | 2.5 | 3 | 4 |
| Hydroxysultaine | | | | | | |
| Quaternium-52 | 2 | 2.5 | 3 | 3.5 | 4 | 5 |
| Polyquaternium-4 | 1.6 | 1.9 | 2.3 | 2.5 | 2.9 | 3 |
| Divinyldimethicone/ | 1 | 1.5 | 2 | 3 | 4 | 5 |
| Dimethicone Copolymer, | | | | | | |
| C12-C13 Pareth-23, C12-C13 | 1.5 | 2 | 2.5 | 3 | 3.5 | 4 |
| Pareth-3 | | | | | | |
| Cocamidopryl PC-Dimonium | 1 | 1.5 | 2 | 3.5 | 4.5 | 5 |
| Chloride Phosphate | | | | | | |
| Diazolidinyl Urea, Iodopropynyl | 1 | 1.5 | 2 | 3 | 4 | 5 |
| Butylcarbamate | | | | | | |
| Fragrance | 0.7 | 1 | 2 | 3 | 4 | 5 |

Example 5 illustrates the various percentage ratio of each ingredient to the remaining ingredients. For example, the percentage ratio of water:remaining ingredients may be 97, i.e. 97:3, meaning that the cleansing formulation comprise 97% water and 3% of the remaining ingredients in the example. Each percentage ratio of each ingredient is an independent value and does not correlate or is in any relationship to the other percentage ratio in the table.

While a number of exemplary embodiments, aspects and variations have been provided herein, those of skill in the art will recognize certain modifications, permutations, additions and combinations and certain sub-combinations of the embodiments, aspects and variations. It is intended that the following claims are interpreted to include all such modifications, permutations, additions and combinations and certain sub-combinations of the embodiments, aspects and variations are within their scope.

What is claimed is:

1. A no-rinse cleansing substrate comprising a composition, wherein the composition comprises:
a) a formulation comprising ingredients of Table 1; or
b) a formulation comprising ingredients of Table 2; or
c) a formulation comprising ingredients of Table 3; or
d) a formulation comprising ingredients of Table 12;
wherein the no-rinse cleansing substrate is used to clean hair.

2. The formulation of claim 1, wherein the formulation comprises ethyl alcohol as a co-solvent, lipophilic agent.

3. The formulation of claim 1, further comprising low molecular weight methyl, isopropyl and butyl alcohol.

4. The formulation of claim 1, further comprising a surface active agent.

5. The formulation of claim 4, wherein the surface active agent is a sulfate free surfactant.

6. The formulation of claim 5, wherein the surface active agent is Cocamindopropyl Hydrosultaine.

7. The formulation of claim 4, wherein the surface active agent is a sulfate based surfactant.

8. The formulation of claim 1, further comprising an anti-static agent.

9. The formulation of claim 8, wherein the anti-static agent comprises quaternium materials that exhibit cationic anti-static properties.

10. The formulation of claim 9, wherein the anti-static agent is Quaternium 52.

11. The formulation of claim 1, further comprising a conditioning agent.

12. The formulation of claim 11, wherein the conditioning agent is Divinyldimethicane/Dimethicone Copolymer.

13. The substrate of claim 1, wherein the substrate comprises material that is nonwoven.

14. The nonwoven material of claim 13, comprising a spunlace of 50% PET and 50% Rayon.

15. The substrate of claim 13, wherein the substrate comprises material selected from a group consisting of PET, Rayon, woodpulp, bamboo rayon, and cotton.

16. The composition of claim 1, wherein a formulation:substrate ratio by weight is about 0.5:1, 1:1, 1.5:1, 2:1, 2.5:1, 3:1, 3.5:1, 4:1, 4.5:1, 5:1, 5.5:1, 6:1, 6.5:1, 7:1, 7.5:1, 8:1, 8.5:1, 9:1, 9.5:1, or 10:1 by weight.

17. The composition of claim 1, further comprising a vitamin complex blend or natural extracts from plants or fragrances.

18. The formulation of claim 1, wherein the formulation comprises one or more ingredients of Table 4, one or more ingredients of Table 5, one or more ingredients of Table 6, one or more ingredients of Table 7, one or more ingredients of Table 8, one or more ingredients of Table 9, one or more ingredients of Table 10, one or more ingredients of Table 11, or one or more ingredients of Table 12.

19. A no-rinse cleansing kit, comprising:
a) the composition of claim 1; and
b) a laminated flexible foil, wherein the foil is a 3-layer foil film comprising PET/AL6/LL60;
wherein the composition of claim 1 is heat-sealed in the laminated flexible foil.

20. A method of treating hair using a no-rinse cleansing substrate of claim 1, the method comprising:
(i) applying the no-rinse cleansing substrate to the hair of a user; and
(ii) allowing the hair to dry.
